(19) United States
(12) Patent Application Publication
(10) Pub. No.: US 2010/0154597 A1
(43) Pub. Date: Jun. 24, 2010

(54) Self contained ink distribution system

(76) Inventor: Joshua T. South, Springfield, OR (US)

Correspondence Address: Joshua T. South, 4830 Elderberry Loop, Springfield, OR, 97478, US

(21) Appl. No.: 12/340,662

(22) Filed: Dec. 20, 2008

(51) Int. Cl. B43K 5/04; B43K 8/00; A61M 37/00

(52) U.S. Cl. 81/9.22; 401/119; 606/186

(57) ABSTRACT

One embodiment of a self contained ink distribution system connected to a tattoo machine (10) is a needle holding tube (12). Surrounding the needle holding tube is a grip (14) which comprises a cavity (11). A passageway (13) leading to a sterile opening (18) is fixedly attached to said cavity. Other embodiments are described and shown.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND

1. Field

This application generally relates to tattooing, more specifically the process of wetting a needle point with ink.

2. Prior Art

The prior art apparatus is illustrated in FIG. 1. To wet the needle points with ink when applying a tattoo, a tattoo artist submerges a tip of a needle holding tube 16 which comprises the needle points, into a hard plastic sterile single use container, in which ink is deposited.

Approximately every 10-20 seconds, the tattooist must stop and re-submerge the needle points in said container, in which ink is deposited.

One draw back to such a tattoo system is the realization that stopping a tattoo procedure to re-submerge the needle points in said container, in which ink is deposited, is tedious and time consuming.

### SUMMARY

In accordance with this embodiment is to allow for the process of wetting a needle point with ink is to be less tedious and more time efficient for the tattoo artist.

### DRAWINGS—FIGURES

FIG. 1 shows a perspective view of prior art device

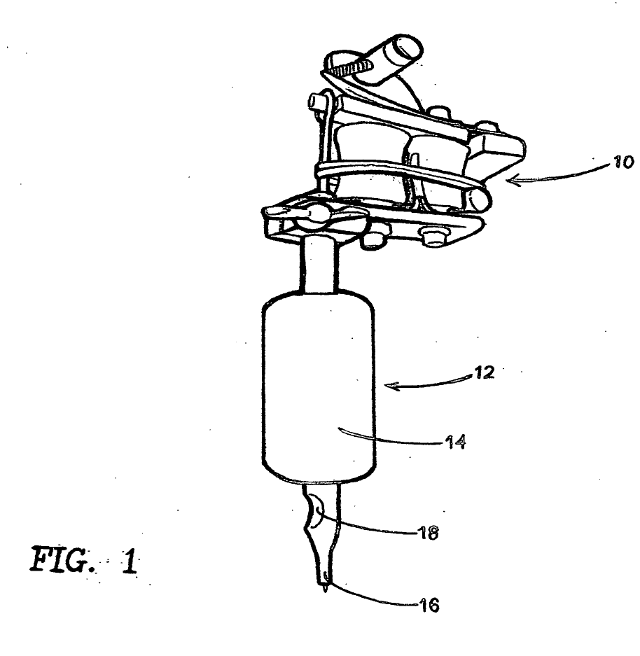

FIG. 2 shows sectional view of the self contained ink distribution system of the present invention.

FIG. 3 shows a sectional side view of the self contained ink distribution system of the present invention.

### DRAWINGS—REFERENCE NUMERALS

10 tattoo machine

11 cavity

12 needle holding tube

13 passageway

14 grip

15 ink container

16 needle holding tube tip

18 sterile opening

### DETAILED DESCRIPTION OF THE PREFERRED EMBODIMENT

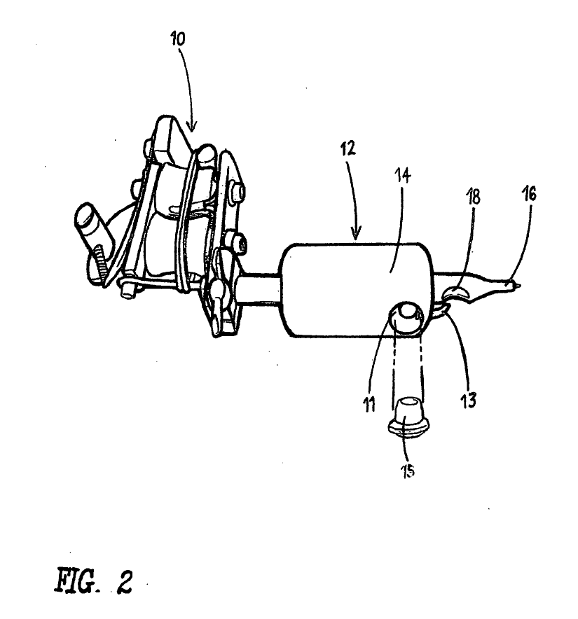

One embodiment of the apparatus is illustrated in FIG. 2. Connected to the tattoo machine 10 is a needle holding tube 12. Surrounding the needle holding tube 10 is a grip 14. In the preferred embodiment the grip 14 comprises a cavity 11. In the preferred embodiment a passageway 13 is mounted to said cavity 11. The passageway 13 leads to a sterile opening 18 of the needle holding tube 12.

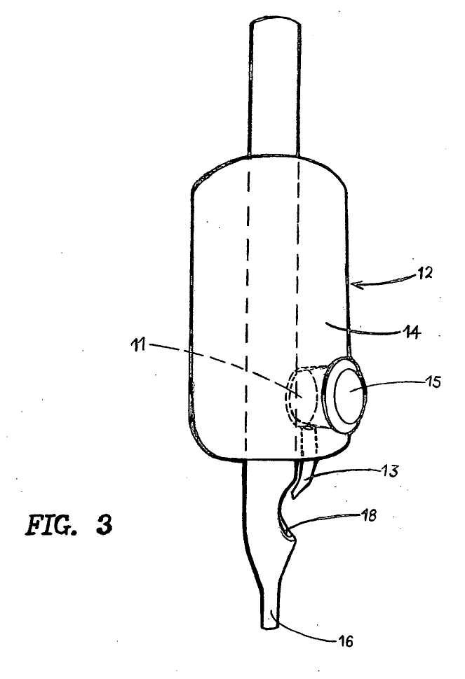

Another embodiment of the apparatus is illustrated in FIG. 2 and FIG. 3. Tattoo pigment is filled into a small single use ink container 15. I contemplate this ink container 15 will be composed of rubber, but could be constructed of some other similar material. More over ink container 15 is the same size and shape of said cavity 11.

Yet another embodiment of the apparatus is illustrated in FIG. 2 and FIG. 3. Said ink container 15 is then inserted into said cavity 11. Where-in due to the forces of air pressure and surface tension the tattoo pigment remains in said cavity 11.

Operation

The operation of the apparatus of this embodiment is as follows: By the force of manual suppression with the thumb or forefinger on the ink container 15, tattoo pigment is directed from the cavity 11 through passageway 13 and a drop is deposited into the sterile opening 18 of the needle holding tube 12. Where-in the forces of gravity carry the tattoo pigment directly to the needle holding tube tip 16.

Advantages

The advantages of the preferred embodiment include but are not limited to, more efficient time management during tattoo procedures. Enables the tattoo artist to reduce the amount of physical exertion and increase the amount of uninterrupted attention during tattoo procedures.

1. A self contained ink distribution system, comprising:
a. a needle holding tube grip having said cavity
b. said passageway is mounted to said cavity
c. said ink container is composed of a flexible material
d. more over said ink container is same size and shape of said cavity
whereby ink can be distributed easily to said needle holding tube tip during tattoo procedures.
